(19) United States
(12) Patent Application Publication
(10) Pub. No.: US 2012/0197366 A1
(43) Pub. Date: Aug. 2, 2012

(54) HIGH DIELECTRIC CONSTANT SHEATH MATERIALS FOR IMPLANTABLE MEDICAL DEVICE LEADS OR CATHETERS

(75) Inventors: Volkert A. Zeijlemaker, Landgraaf (NL); Edze J. Tijsma, Middelburg (NL)

(73) Assignee: MEDTRONIC, INC., Minneapolis, MN (US)

(21) Appl. No.: 13/037,116

(22) Filed: Feb. 28, 2011

Related U.S. Application Data
(60) Provisional application No. 61/437,389, filed on Jan. 28, 2011.

(51) Int. Cl. A61N 1/05; H01R 43/20

(52) U.S. Cl. 607/116; 29/876

(57) ABSTRACT

A medical lead and a method of making the medical lead, the medical lead having a proximal end for electrical connection to a medical device and a distal end implantable proximate a target tissue. The lead includes a conductor and a sheath disposed over the conductor, the sheath comprising a polymer and a filler mixed in the polymer. The filler has a dielectric constant that is different from a dielectric constant of the polymer. The weight percentage of the filler in the sheath is selected such that energy induced by an external disruptive energy field in the conductor that is absorbed by the target tissue proximate the distal end of the lead is below a predetermined threshold energy.

This application claims the benefit of U.S. Provisional Application No. 61/437,389, filed on Jan. 28, 2011, the entire content of which is incorporated herein by reference.

### TECHNICAL FIELD

The present disclosure relates to catheters or leads for implantable medical devices.

### BACKGROUND

Some types of medical devices provide therapeutic electrical stimulation to tissue of a patient via electrodes carried on one or more implantable leads and/or monitor electrical signals of the tissue via the electrodes carried on the one or more implantable leads. The electrical stimulation or sensing signals are carried to or from the electrodes by conductors within the leads. Medical devices, which may or may not be implantable, may comprise cardiac pacemakers, cardioverters, defibrillators, neurostimulators, muscle stimulators, or the like. In the case of therapeutic electrical stimulation, the electrical stimulation may be delivered to the tissue via the electrodes of the implantable leads in the form of neurostimulation pulses, pacing pulses, cardioversion shocks, defibrillation shocks, cardiac resynchronization signals, or other signals. In some cases, electrodes carried by the implantable leads may be used to sense one or more physiological signals to monitor the condition of a patient and/or to control delivery of therapeutic electrical stimulation based on the sensed signals.

The leads and the medical device may be exposed to an external energy field, such as a magnetic or radiofrequency (RF) field, for any of a number of reasons. The external energy field may, in some instances, interfere with or otherwise disrupt the intended operation of the lead or the medical device. Such an external energy field is referred to herein as a disruptive energy field. For example, one or more medical procedures may need to be performed on the patient upon whom the medical device acts for purposes of diagnosis or therapy. For example, the patient may need to have a magnetic resonance imaging (MRI) scan, electrocautery, diathermy or other medical procedure that produces a magnetic field, an electromagnetic field, an electric field or other external disruptive energy field.

The disruptive energy field may induce energy on the conductors of one or more of the implantable leads coupled to the IMD. In some examples, the induced energy may produce lead heating that may cause unwanted heating of the patient's tissue.

### SUMMARY

In general, the present disclosure is related to an implantable catheter or lead used with a medical device having a sheath formed from high dielectric constant materials. The sheath materials may comprise a filler mixed within a polymer. The filler may have a dielectric constant different from the dielectric constant of the polymer so that the overall effective dielectric constant of the sheath is different from that of the polymer alone. The change in overall effective dielectric constant allows for tuning of a characteristic electrical impedance of the catheter or lead such that energy induced in the lead by a disruptive energy field may be reflected from a distal end of the catheter or lead rather than absorbed by patient tissue at the distal end.

In one example, the present disclosure is directed to a method comprising forming a medical lead having a proximal end for electrical connection to a medical device and a distal end implantable proximate a target tissue, the lead comprising a conductor and a sheath disposed over the conductor, the sheath comprising a polymer and a filler mixed in the polymer, wherein the filler has a dielectric constant that is different from a dielectric constant of the polymer, and selecting the weight percentage of the filler in the sheath such that energy induced by an external disruptive energy field in the conductor that is absorbed by the target tissue proximate the distal end of the lead is below a predetermined threshold energy

In another embodiment, the disclosure is directed to a lead for a medical device, the lead comprising a conductor having a proximal end for electrically connecting to a medical device and a distal end implantable proximate a target tissue, and a sheath disposed over the conductor, the sheath comprising a polymer and a filler mixed in the polymer, wherein the filler has a dielectric constant that is different from a dielectric constant of the polymer, and wherein the weight percentage of the filler in the sheath is selected such that an amount of energy induced by an external disruptive energy field in the conductor that exceeds a predetermined threshold energy is reflected away from the distal end of the lead.

In another embodiment, the disclosure is directed to a system comprising a medical device, and a medical lead having a proximal end electrically coupled to the medical device and a distal end implantable proximate a target tissue, the lead comprising a conductor and a sheath disposed over the conductor, the sheath comprising a polymer and a filler mixed in the polymer, wherein the filler has a dielectric constant that is different from a dielectric constant of the polymer, wherein the weight percentage of the filler in the sheath is selected such that an amount of energy induced by an external disruptive energy field in the conductor that exceeds a predetermined threshold energy is reflected away from the distal end of the lead.

This summary is intended to provide an overview of the subject matter described in this disclosure. It is not intended to provide an exclusive or exhaustive explanation of the techniques as described in detail within the accompanying drawings and description below. The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a conceptual diagram illustrating an example therapy system comprising an implantable medical device for the delivery of cardiac stimulation therapy to a patient via implantable leads.

FIG. 2 is a conceptual diagram further illustrating the implantable medical device and leads of the system of FIG. 1 in greater detail.

FIG. 3 is a longitudinal cross-sectional view showing a portion of a lead that may be used with a therapy system.

FIG. 4 is a transverse cross-sectional view showing the lead across line 4-4 in FIG. 3.

FIG. 5 is a transverse cross-section view showing another lead that may be used with a therapy system.

FIG. 6 is a flow diagram illustrating an example method of forming a lead that may be used with a therapy system.

FIG. 7 is a flow diagram illustrating another example method of forming a lead that may be used with a therapy system.

### DETAILED DESCRIPTION

In general, this disclosure is directed to techniques for configuring an implantable catheter or lead usable with a medical device, such as an implantable medical device (IMD), such that lead heating is controlled and/or reduced when the catheter or lead is subject to a disruptive energy field. The catheter/lead and medical device may form part of an implantable medical system for delivery of therapy to a patient and/or sensing of signals from the patient. In some examples, the disruptive energy field may be a magnetic or radio frequency (RF) field generated by an electromagnetic energy source. In additional examples, the disruptive energy field may be an energy field produced by a medical imaging modality, such as a magnetic resonance imaging (MRI) modality. The energy from the disruptive energy field may induce current flow within one or more conductors in the lead, which can produce lead heating, particularly at electrodes coupled to the conductors. As used herein, the terms “lead heating” or “lead heating effects” may refer to the heating of tissue proximate to one or more electrodes coupled to an implantable medical device.

When an implantable catheter or lead is placed within the presence of a disruptive energy field, a conductor within the catheter or lead may act as an antenna such that the electromagnetic waves and/or energy associated with the field may induce a current that propagates along the length of the catheter or lead. Some of the energy may propagate in the direction of a distal end of the catheter or lead. Electrodes used for therapy delivery or sensing are generally located at or proximate to the distal end of the catheter or lead. A proximal end of the catheter or lead may be coupled to a medical device housing, which may contain stimulation generation circuitry, sensing circuitry, and/or other circuitry.

In certain conditions, the energy may propagate from the conductor to electrodes coupled to the conductor where the energy may be dissipated as heat that is absorbed by the patient's tissue. For example, when the electrical characteristic impedance of the catheter or lead and electrodes substantially match the tissue impedance, electrical energy may be absorbed by the tissue through the electrodes in the form of heat. Because the electrodes may be very small, e.g., less than 2 mm², the heat dissipation may cause tissue temperatures to rise beyond that which is desirable. However, in some other conditions, the catheter or lead may be configured so that energy above a certain threshold is reflected back up the catheter or lead toward the medical device housing coupled to the proximal end of the lead. For example, the electrical characteristic impedance of the catheter or lead may be tuned, such as so that the impedance of the catheter or lead is mismatched with the impedance of the patient's tissue, so that energy above the threshold is reflected at the distal end rather than absorbed by the patient's tissue. The energy may be more effectively dissipated at the medical device housing without causing appreciable tissue heating, e.g., because the housing may have a much larger surface area for dissipating heat, relative to the electrode, resulting in a small temperature change in the surrounding tissue.

The electromagnetic energy source may produce electromagnetic energy at any frequency. In examples where the electromagnetic energy source is an MRI modality, the electromagnetic energy source may, in some examples, produce electromagnetic waves or energy (e.g., a disruptive energy field) having frequencies of 42 MHz, 64 MHz, and/or 128 MHz. The proportion of 42 MHz radio frequency (RF) to a 1 Tesla (T) static magnetic field is controlled by the Larmar frequency. Therefore 42 MHz, 64 MHz, and 128 MHz correspond to 1 T, 1.5 T, and 3 T MRI modalities, respectively. However, the techniques in this disclosure may be applied to other frequencies of MRI modalities as well.

In some examples, the present disclosure is directed to a sheath for covering an implantable catheter or lead usable with a medical device, such as an implantable medical device (IMD). The sheath may provide for tuning of the characteristic electrical impedance of the catheter or lead in order to tune coupling between the lead and patient tissue surrounding the lead. The characteristic impedance may be tuned so that induced energy that propagates down the catheter or lead toward a distal end above a predetermined threshold amplitude of the energy may be reflected from the distal end in order to reduce the likelihood of tissue heating that may result from high-energy radiofrequency (RF) electromagnetic fields, such as the fields associated with magnetic resonance imaging (MRI). In other words, the sheath of the present disclosure may be configured so that the amount of energy that is absorbed by the tissue from the electrodes in the form of heat is below a selected threshold such that the remaining energy is reflected from the distal end back toward the proximal end, e.g., so that the energy may be dissipated at the medical device. The characteristic impedance may be tuned by modulating the dielectric constant (εᵣ, also referred to as the relative permittivity) of the sheath by adding a filler material to the polymer that comprises the sheath, wherein the filler has a dielectric constant, ε_{r, filler}, that is different from the dielectric constant of the polymer, ε_{r, polymer}.

In some examples, the amount of energy propagated along the catheter or lead toward the distal end, i.e., the “propagated energy,” may refer to a raw amount of propagated energy. As used herein, a raw amount of propagated energy may refer to the magnitude of electromagnetic energy that is propagated toward the distal end of the catheter or lead in response to electromagnetic energy being induced in the catheter or lead by a disruptive electromagnetic field. In some examples, the raw amount of propagated energy may be an electromagnetic wave that travels towards the distal end of the catheter or lead. In some instances, the energy may be propagated as a current.

In additional examples, the amount of energy propagated along the catheter or lead toward the distal end may refer to a composite amount of propagated energy. As used herein, a composite amount of propagated energy may refer to the combination (e.g., superposition) of the raw amount of propagated energy along the catheter or lead toward the distal end and additional amounts of energy reflected from a proximal end (device side) along the respective catheter or lead. The additional amounts of energy may include energy that is reflected at the proximal end of the catheter or lead, e.g., energy reflected by the medical device. In some examples, the additional amounts of energy may include energy that is induced in the respective electrical catheter or lead by a disruptive electromagnetic energy field.

For example, when an implantable catheter or lead is subject to a disruptive electromagnetic energy field, the disruptive electromagnetic energy field may induce a first type of electromagnetic wave that may travel along the catheter or lead towards the proximal end of the catheter or lead (e.g., towards the interface between the lead or catheter and the medical device). The disruptive electromagnetic energy field may also induce a second type of electromagnetic wave in the catheter or lead that may travel along the catheter or lead towards the distal end of the catheter or lead. The medical device may reflect some of the first type of electromagnetic wave to produce a third type of electromagnetic wave. For example, an electrical network formed by components of the medical device may reflect a portion of the first type of electromagnetic wave and produce the third type of electromagnetic wave. The third type of electromagnetic wave may travel along the catheter or lead towards the distal end of the catheter or lead (e.g., away from the interface between the catheter or lead and the medical device). The second type of electromagnetic wave and the third type of electromagnetic may combine (e.g., superpose) to form a composite amount of propagated energy. Thus, in such examples, the composite amount of propagated energy may refer to the combination (e.g., superposition) of the second type of electromagnetic wave and the third type of electromagnetic wave. The techniques in this disclosure may, in some examples, be used to cause the portion of the composite amount of energy propagated along the catheter or lead in response to energy produced by an electromagnetic energy source to be below a predetermined threshold, wherein the energy above the predetermined threshold is reflected from the distal end back toward the medical device.

For purposes of illustration, the techniques of this disclosure will be described with respect to a disruptive energy field generated by an imaging modality and, more specifically, a magnetic resonance imaging (MRI) modality. The techniques of this disclosure may, however, be used in the context of other disruptive energy fields generated by imaging modalities other than MRI modalities or non-imaging medical or non-medical devices that generate an energy field.

Although the techniques in this disclosure are described with respect to “energy” or “an amount of energy,” it should be recognized that other measures of electromagnetic fields and/or radiation may also be used. For example, “power” or “an amount of power” may be used in place of “energy” and/or “an amount of energy.” In addition, where this disclosure refers to “electromagnetic waves,” the disclosure may also be referring to “electromagnetic energy” and/or “electromagnetic radiation.”

The examples shown and discussed with respect to the following Figures are generally described as corresponding to a lead for carrying a conductor and electrodes in order to provide electrical stimulation and/or sensing of a target tissue. Therefore, the remainder of the specification will be described with respect to “leads.” However, “lead,” as used herein, may also refer to a catheter that provides for the delivery of a fluid to the target tissue. For example, a catheter may comprise an interior lumen for the delivery of a therapeutic agent to the target tissue, and the catheter may also comprise a conductive material, such as one or more conductors for carrying electrical stimulation signals to the target tissue. The catheter may also comprise one or more electrodes for delivery of electrical stimulation to a target tissue and/or for sensing bioelectric signals proximate the target tissue, wherein the electrode(s) are electrically coupled to the conductor.

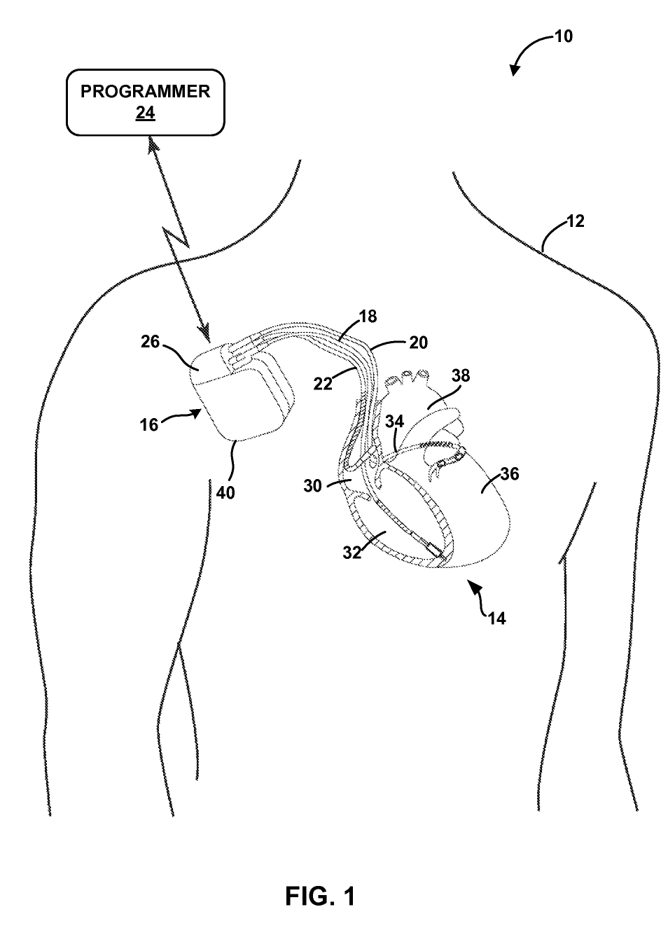

FIG. 1 is a conceptual diagram illustrating an example therapy system 10 that may be used to provide therapy to a patient 12. Patient 12 ordinarily, but not necessarily, will be a human. Therapy system 10 may include an implantable medical device (IMD) 16, such as an implantable cardiac device, and a programmer 24. In the example depicted in FIG. 1, IMD 16 is connected (or “coupled”) to leads 18, 20, and 22 via a connector block 26. IMD 16 may be, for example, a device that provides cardiac rhythm management therapy, and may include, for example, an implantable pacemaker, cardioverter, and/or defibrillator that provides therapy to heart 14 of patient 12 via electrodes coupled to one or more of leads 18, 20, and 22. In some examples, IMD 16 may deliver pacing pulses, but not cardioversion or defibrillation shocks, while in other examples, IMD 16 may deliver cardioversion or defibrillation shocks, but not pacing pulses. In addition, in further examples, IMD 16 may deliver pacing pulses, cardioversion shocks, and defibrillation shocks.

Leads 18, 20, 22 that are coupled to IMD 16 may extend into the heart 14 of patient 12 to sense electrical activity of heart 14 and/or deliver electrical stimulation to heart 14. In the example shown in FIG. 1, right ventricular (RV) lead 18 extends through one or more veins (not shown), the superior vena cava (not shown), and right atrium 30, and into right ventricle 32. Left ventricular (LV) coronary sinus lead 20 extends through one or more veins, the vena cava, right atrium 30, and into the coronary sinus 34 to a region adjacent to the free wall of left ventricle 36 of heart 14. Right atrial (RA) lead 22 extends through one or more veins and the vena cava, and into the right atrium 30 of heart 14. In other examples, IMD 16 may deliver stimulation therapy to heart 14 by delivering stimulation to an extravascular tissue site in addition to or instead of delivering stimulation via electrodes of intravascular leads 18, 20, 22.

IMD 16 may sense electrical signals attendant to the depolarization and repolarization of heart 14 (e.g., cardiac signals) via electrodes (not shown in FIG. 1) coupled to at least one of the leads 18, 20, 22. In some examples, IMD 16 provides pacing pulses to heart 14 based on the cardiac signals sensed within heart 14. The configurations of electrodes used by IMD 16 for sensing and pacing may be unipolar or bipolar. IMD 16 may also provide defibrillation therapy and/or cardioversion therapy via electrodes located on at least one of the leads 18, 20, 22. IMD 16 may detect arrhythmia of heart 14, such as fibrillation of ventricles 32 and 36, and deliver defibrillation therapy to heart 14 in the form of electrical shocks. In some examples, IMD 16 may be programmed to deliver a progression of therapies, e.g., shocks with increasing energy levels, until a fibrillation of heart 14 is stopped. IMD 16 may detect fibrillation by employing one or more fibrillation detection techniques known in the art. For example, IMD 16 may identify cardiac parameters of the cardiac signal, e.g., R-waves, and detect fibrillation based on the identified cardiac parameters.

In some examples, programmer 24 may be a handheld computing device or a computer workstation. Programmer 24 may include a user interface that receives input from a user. The user interface may include, for example, a keypad and a display, which may be, for example, a cathode ray tube (CRT) display, a liquid crystal display (LCD) or light emitting diode (LED) display. The keypad may take the form of an alphanumeric keypad or a reduced set of keys associated with particular functions. Programmer 24 can additionally or alternatively include a peripheral pointing device, such as a mouse, via which a user may interact with the user interface. In some embodiments, a display of programmer 24 may include a touch screen display, and a user may interact with programmer 24 via the display.

A user, such as a physician, technician, or other clinician, may interact with programmer 24 to communicate with IMD 16. For example, the user may interact with programmer 24 to retrieve physiological or diagnostic information from IMD 16. A user may also interact with programmer 24 to program IMD 16, e.g., select values for operational parameters of IMD 16.

Programmer 24 may communicate with IMD 16 via wireless communication using any techniques known in the art. Examples of communication techniques may include, for example, low frequency or radiofrequency (RF) telemetry, but other techniques are also contemplated. In some examples, programmer 24 may include a programming head that may be placed proximate to the patient's body near the IMD 16 implant site in order to improve the quality or security of communication between IMD 16 and programmer 24.

Each lead 18, 20, 22 may include one or more electrical conductors. Each conductor may extend between an electrical contact at a proximal end of a lead and an electrode at a distal end of the lead. The conductors may conduct stimulation current to the electrodes and/or conduct sensing current from the electrodes. The electrical contacts may be electrically coupled to respective electrical terminals in a header associated with housing 40 of the IMD 16, such as within connector block 26. The ribbon bonding and/or conductive traces may electrically couple the electrical terminals to corresponding electrical terminals inside the housing of the IMD 16, e.g., on a circuit board that includes various electronic circuit components for generation, control and/or processing of electrical stimulation and/or sensing signals. The conductors may carry current that is induced by energy associated with an electromagnetic field, such as an MRI or other imaging modality.

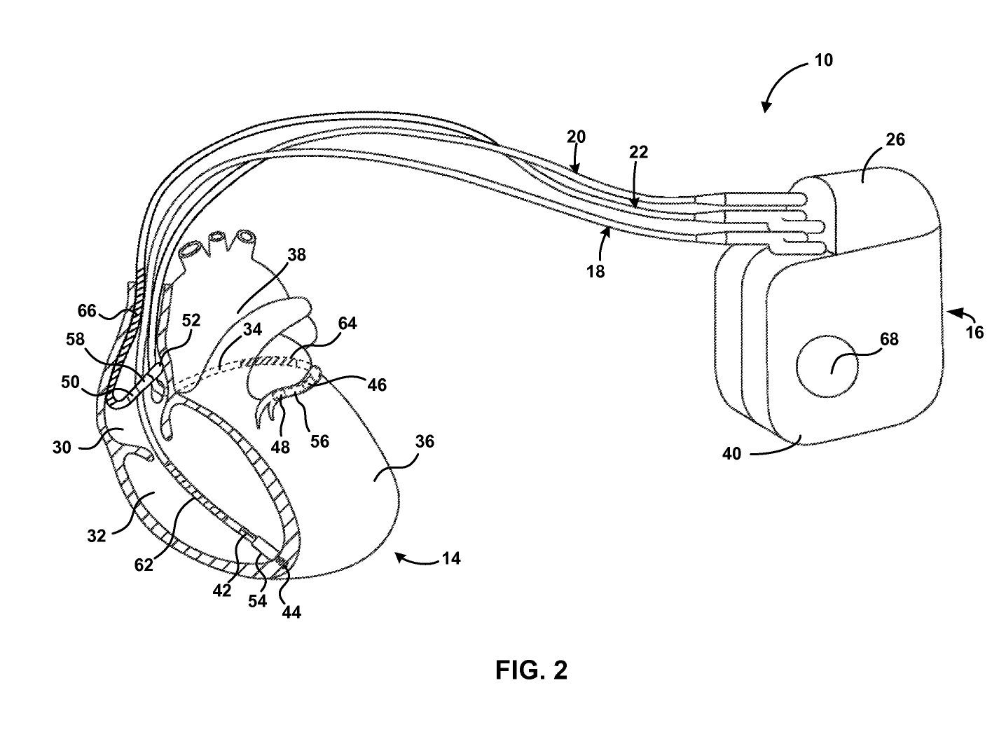

FIG. 2 is a conceptual diagram illustrating a three-lead IMD 16 and leads 18, 20 and 22 of therapy system 10 in greater detail. Leads 18, 20, 22 may be electrically coupled to a signal generator and a sensing module of IMD 16 via connector block 26. In some examples, proximal ends of leads 18, 20, 22 may include electrical contacts that electrically couple to respective electrical contacts within connector block 26 of IMD 16. In addition, in some examples, leads 18, 20, 22 may be mechanically coupled to connector block 26 with the aid of set screws, connection pins, snap connectors, or another suitable mechanical coupling mechanism.

Each of the leads 18, 20, 22 may carry a number of concentric coiled conductors separated from one another by tubular insulative sheaths. Other lead configurations are also contemplated, such as configurations that do not include coiled conductors. In the illustrated example, bipolar electrodes 42 and 44 are located proximate to a distal end of lead 18 in right ventricle 32. In addition, bipolar electrodes 46 and 48 are located proximate to a distal end of lead 20 in left ventricle 36 and bipolar electrodes 48 and 52 are located proximate to a distal end of lead 22 in right atrium 30. Although no electrodes are located in left atrium 38 in the illustrated example, other examples may include electrodes in left atrium 38.

Electrodes 42, 46, and 50 may take the form of ring electrodes, and electrodes 44, 48, and 52 may take the form of extendable helix tip electrodes mounted retractably within insulative electrode heads 54, 56, and 58, respectively. In other examples, one or more of electrodes 42, 46, and 50 may take the form of small circular electrodes proximate to a tip of a tined lead or other fixation element. Leads 18, 20, 22 may also include elongated electrodes 62, 64, 66, respectively, which may take the form of a coil. Each of the electrodes 42, 44, 46, 48, 50, 52, 62, 64, and 66 may be electrically coupled to a respective one of the conductors within its associated lead 18, 20, 22, and thereby coupled to respective ones of the electrical contacts on the proximal end of leads 18, 20, 22.

In some examples, as illustrated in FIG. 2, IMD 16 includes one or more housing electrodes, such as housing electrode 68, which may be formed integrally with an outer surface of housing 40 of IMD 16 or otherwise coupled to housing 40. In some examples, housing electrode 68 is defined by an uninsulated portion of an outward facing portion of IMD housing 40. Other divisions between insulated and uninsulated portions of housing 40 may be employed to define two or more housing electrodes. In some examples, housing electrode 68 comprises substantially all of housing 40. Housing 40 may enclose a signal generator that generates therapeutic stimulation, such as cardiac pacing pulses and/or cardioversion/defibrillation shocks, as well as a sensing module for monitoring the rhythm of heart 12.

In some examples, IMD 16 may sense electrical signals attendant to the depolarization and repolarization of heart 14 via electrodes 42, 44, 46, 48, 50, 52, 62, 64, and 66. The electrical signals are conducted to IMD 16 from the electrodes via the respective leads 18, 20, 22 or, in the case of housing electrode 68, a conductor coupled to housing electrode 68. IMD 16 may sense such electrical signals via any bipolar combination of electrodes 42, 44, 46, 48, 50, 52, 62, 64, and 66. Furthermore, any of the electrodes 42, 44, 46, 48, 50, 52, 62, 64, and 66 may be used for unipolar sensing in combination with housing electrode 68.

Any multipolar combination of two or more of electrodes 42, 44, 46, 48, 50, 52, 62, 64, and 66 may be considered a sensing electrode configuration. Usually, but not necessarily, a sensing electrode configuration is a bipolar electrode combination on the same lead, such as electrodes 42 and 44 of lead 18. On one lead having three electrodes, there may be at least three different sensing electrode configurations available to IMD 16. These sensing electrode configurations are, for the example of lead 18, tip electrode 44 and ring electrode 43, tip electrode 44 and elongated electrode 62, and ring electrode 42 and elongated electrode 62. However, some examples may utilize sensing electrode configurations having electrodes of two different leads. Further, a sensing electrode configuration may utilize housing electrode 68, which may provide a unipolar sensing electrode configuration. In some examples, a sensing electrode configuration may comprise multiple housing electrodes 68. In any sensing electrode configuration, the polarity of each electrode in the may be configured as appropriate for the application of the sensing electrode configuration.

In some examples, IMD 16 delivers pacing pulses via bipolar combinations of electrodes 42, 44, 46, 48, 50, and 52 to produce depolarization of cardiac tissue of heart 14. In some examples, IMD 16 delivers pacing pulses via any of electrodes 42, 44, 46, 48, 50, and 52 in combination with housing electrode 68 in a unipolar configuration. Furthermore, IMD 16 may deliver cardioversion or defibrillation shocks to heart 14 via any combination of elongated electrodes 62, 64, 66, and housing electrode 68. Electrodes 62, 64, 66, and 68 may also be used to deliver cardioversion shocks to heart 12. Electrodes 42, 44, 46, 48, 50, 52, 62, 64, and 66 may be fabricated from any suitable electrically conductive material, such as, but not limited to, platinum, platinum alloy, Titanium nitride or other materials known to be usable in implantable defibrillation electrodes.

As described above, exposure of IMD 16 to a disruptive energy field, e.g., one or more fields produced by an MRI imaging modality, may result in lead heating, particularly at electrodes 42, 44, 46, 48, 50, 52, 62, 64, and 66. For example, RF fields produced by an MRI imaging modality may induce energy on one or more conductors of respective ones of implantable leads 18, 20, or 22 or on the housing electrode, and some of the induced energy may propagate toward the distal end of lead 18, 20, or 22. The energy may also propagate from the conductor to electrodes connected to the conductors, such as electrodes 42, 44, 46, 48, 50, 52, 62, 64, and 66, where the energy may be dissipated to the tissue in the form of heat. As noted above, electrodes 42, 44, 46, 48, 50, 52, 62, 64, and 66 may have a small surface area upon which to dissipate the heat such that electrodes 42, 44, 46, 48, 50, 52, 62, 64, and 66 may be heated to undersirably high temperatures. According to this disclosure, a material of an outer sheath of each implantable lead 18, 20, or 22 may be provided that may reduce and/or control the absorption of energy in the tissue of patient 12 by tuning the characteristic impedance of the lead 18, 20, 22 so that propagated energy beyond a particular, predetermined threshold is reflected from the distal end of the lead 18, 20, 22 back toward the proximal end, where, for example, it may be dissipated at IMD 16, which has a larger surface area to dissipate heat without undesirably heating of the tissue of patient 12.

The configuration of therapy system 10 and leads 18, 20, 22 illustrated in FIGS. 1 and 2 is merely one example. In other examples, a lead in accordance with the present disclosure may be used with other types of medical devices. Moreover, a lead in accordance with the present disclosure may be used as a cardiac lead, an endocardial lead, a neurological lead, a patch electrode, or a catheter. In addition, while the leads themselves are at least partially implantable within patient 12, and although the present disclosure describes that leads 18, 20, 22 are used in conjunction with an implantable medical device 16, the medical device to which the leads are coupled need not be implantable within patient 12. In examples in which housing 40 is not implanted in patient 12, IMD 16 may deliver electrical stimulation and/or other therapies to heart 14 or other tissue via percutaneous leads that extend through the skin of patient 12 to a variety of positions within or outside of heart 14. Percutaneous leads additionally or alternatively may be used for sensing.

In other examples of therapy systems that provide electrical stimulation therapy to heart 14, a therapy system may include any suitable number of leads coupled to IMD 16, and each of the leads may extend to any location within or proximate to heart 14. For example, a therapy system may include a single chamber or dual chamber device rather than a three-chamber device as shown in FIGS. 1 and 2. In a single chamber configuration, IMD 16 may be electrically connected to a single lead that includes stimulation and sense electrodes within left ventricle 36. In one example of a dual chamber configuration, IMD 16 may be electrically connected to a single lead that includes stimulation and sense electrodes within left ventricle 36 as well as sense and/or stimulation electrodes within right atrium 30. In another example of a dual chamber configuration, IMD 16 may be connected to two leads that extend into a respective one of the right atrium 30 and left ventricle 36. Other lead configurations are contemplated, and the techniques in this disclosure are not limited to any particular number of leads or configuration of leads.

The techniques of this disclosure may be used to operate an IMD that provides other types of electrical stimulation therapy other than cardiac rhythm management therapy or in devices that provide no therapy at all, but only monitor a condition of a patient. For example, the IMD may be a device that provides electrical stimulation to a tissue site of patient 12 proximate a muscle, organ or nerve, such as a tissue proximate a vagus nerve, spinal cord, brain, stomach, pelvic floor or the like. For example, a lead or leads may be coupled to a drug delivery device, a monitoring device that monitors one or more physiological parameter of patient 12, a neurostimulator (e.g., a spinal cord stimulator, a deep brain stimulator, a pelvic floor stimulator, a peripheral nerve stimulator, or the like), or the like. Moreover, the techniques may be used to operate an IMD that provides other types of therapy, such as drug delivery or infusion therapies. As such, description of these techniques in the context of cardiac rhythm management therapy should not be limiting of the techniques as broadly described in this disclosure.

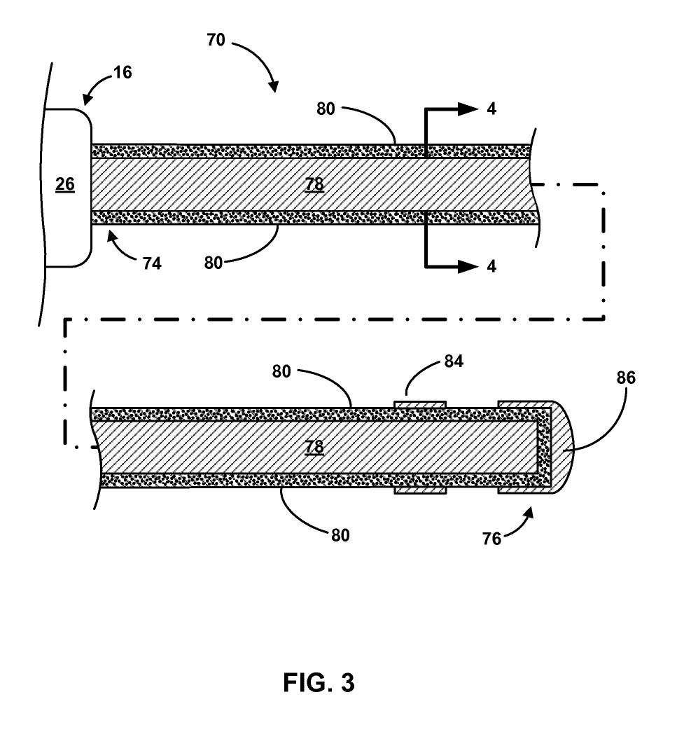

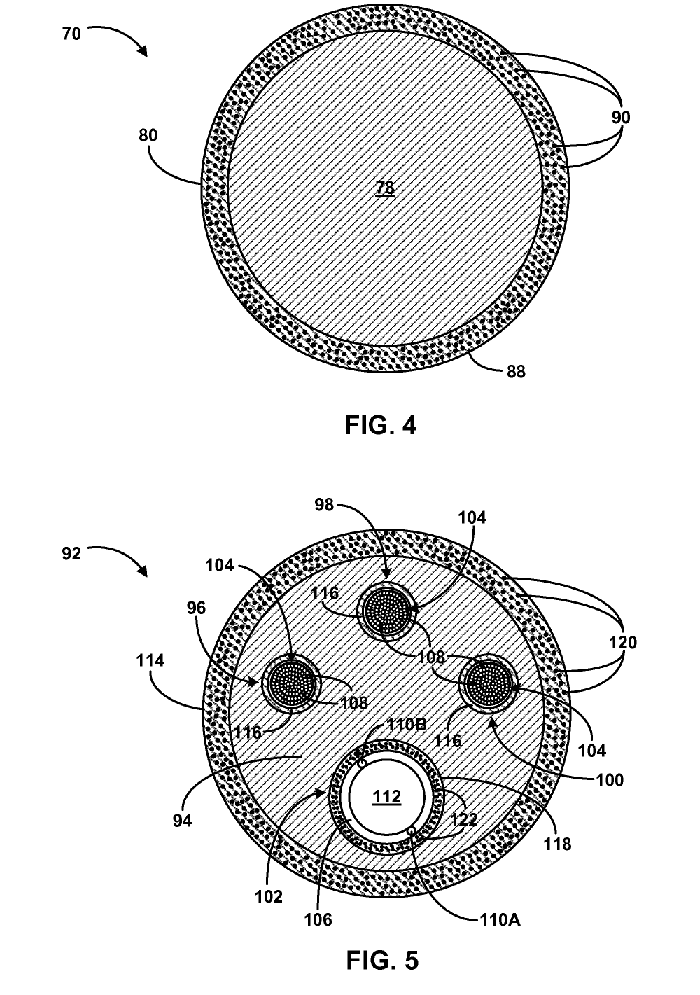

FIGS. 3 and 4 show an example lead 70 in accordance with the present disclosure. FIG. 3 is a longitudinal cross-sectional view of lead 70, while FIG. 4 is a transverse cross-sectional view taken along line 4-4 in FIG. 3. Lead 70 may be used as any of the leads described in the present disclosure, including any of leads 18, 20, 22 described with respect to FIGS. 1 and 2. In one example, lead 70 extends from a proximal end 74 to a distal end 76. Proximal end 74 may be electrically coupled to a medical device, such as to IMD 16 via connector block 26, and distal end 76 is implantable proximate to a target tissue of patient 12 such that the medical device may provide electrical stimulation to the target tissue of patient 12 and/or provide for sensing of bioelectric signals proximate the target tissue.

Lead 70 comprises one or more conductors 78 that extend from proximal end 74 to distal end 76 and a sheath 80 disposed over conductor 78. In some examples, sheath 80 may be an outer surface of lead 70. In other words, sheath 80 may be viewed as forming a body of lead 70 within which conductor 78 (and possibly other conductors) resides. An additional layer, such as an insulation layer, may be disposed between conductor 78 and sheath 80 (not shown). For example, conductor 78 may include and insulative jacket surrounding conductor 78. In some examples, conductor 78 runs along the center of lead 70, e.g., such that conductor 78 and sheath 80 are substantially concentric within lead 70, as shown in FIG. 4. In other examples, sheath 80 may not form the body of lead 70. Instead, sheath 80 may be viewed as a coating or jacket surrounding conductor 78 and lead 70 may have a separate lead body that may or may not have a filler mixed therein, e.g., as described with respect to the example illustrated in FIG. 5.

Electrical stimulation signals and/or sensing signals are carried by conductor 78 extending through an interior of lead 70. One or more electrodes 84, 86 may be carried on lead 70 proximate distal end 76, wherein electrodes 84, 86 provide for the electrical stimulation of the target tissue of patient 12 or provide for the sensing of bioelectric signals at the target tissue. Conductor 78 may also provide an electrical conduction pathway for stimulation pulses to be transmitted from a signal generator within IMD 16 to an electrode, such as electrode 84 or 86, proximate distal end 76, such as when IMD 16 provides cardiac pacing or defibrillation. Although FIGS. 3 and 4 show only a single conductor 78, other examples may comprise two or more conductors, wherein each conductor provides a conduction pathway to a specific electrode 84, 86. For example, a first conductor may be electrically coupled to electrode 84, and a second conductor may be electrically coupled to electrode 86. Conductor 78 may also provide a conduction pathway from a corresponding electrode 84, 86 to a sensing module within IMD 16 to determine a condition of patient 12, such as when IMD 16 senses the rhythm of heart 14 to determine if a particular type of therapy is needed. Each electrode 84, 86 is electrically coupled to a conductor (not shown), such as conductor 78 so that signals may be transmitted between conductor 78 and the corresponding electrode 84, 86. In some examples, electrodes 84, 86 may have any of the configurations described above with respect to leads 18, 20, 22 shown in FIG. 2. For example, electrode 84 in FIG. 3 is a ring electrode 84 proximate distal end 76, while electrode 86 is a tip electrode 86 placed at distal end 76. Other electrode configurations may be used, such as ring electrodes 42, 46, 50, tip electrodes 48, 52, helical tip electrode 44, or elongated electrodes 62, 64, 66, as described above with respect to FIG. 2.

Conductor 78 is shown as a generally cylindrical body in FIGS. 3 and 4. However, other configurations are possible, such as a ribbon or coiled conductor. In one example, a coiled conductor comprises a coiled conductor extending along the length of lead 70. The coiled conductor may comprise a closely wound, generally helical wire having a winding pitch and outer diameter selected to provide desired characteristics to the conductor, such as durability or impedance.

Conductor 78 comprises an electrically conductive material for the transmission of electrically energy longitudinally along conductor 78. Conductor 78 may be fabricated from any suitable electrically conductive material, including, but not limited to, platinum, platinum alloy, titanium nitride, tantalum, stainless steel, gold, iridium, tungsten, or any other materials that are known to be usable as a conductor for the transmission of electrical stimulation or for the transmission of sensed electrical signals. In one example, conductor 78 comprises a biocompatible material. Similarly, electrodes 84, 86 comprise an electrically conductive material capable of transmitting electrical energy to the target tissue and/or for sensing bioelectric signals from the target tissue. Electrodes 84, 86 may be made from any suitable electrically conductive material, including, but not limited to, platinum, platinum alloy, titanium nitride, tantalum nitride, or any other materials that are known to be usable as electrodes for the transmission of electrical stimulation or for the transmission of sensed electrical signals. In one example, electrodes 84, 86 comprise a biocompatible material.

In some examples, sheath 80 comprises a polymer 88 and a filler 90 mixed in polymer 88 (best seen in FIG. 4). In some examples, polymer 88 comprises a generally electrically insulating material that is formed over conductor 78. In some examples, polymer 88 is a generally flexible and resilient material capable of withstanding the process of implanting lead 70 within patient 12, e.g., of being directed through one or more blood vessels as distal end 76 is implanted within heart 14 of patient 12. Examples of materials that may be used as polymer 88 of sheath 80 include, but are not limited to, polyurethanes, polyamides, polyesters, silicones, silicone rubbers, polyisoprenes, butadiene based polymers or copolymers, polyethylenes, polypropylenes, and vinyl based polymers or copolymers, such as an ethylene-vinyl acetate copolymer. In one example, a biocompatible industrial polymer, such as the ethylene-vinyl acetate copolymer sold under the trade name ELVAX 240, manufactured by E.I. du Pont de Nemours and Co., Inc., Wilmington, Del. As noted above, polymer 88 is generally electrically insulating, and in some examples, polymer 88 has a dielectric constant, ε_{r, polymer}, of between about 1 and about 10, and more particularly, for example, between about 3 and about 7. For example, a silicone rubber may have a dielectric constant, ε_{r, polymer}, of between about 3 and about 10, polyurethane may have a dielectric constant, ε_{r, polymer}, of about 7, polyisoprene may have a dielectric constant, ε_{r, polymer}, of about 2.4, polybutadiene may have a dielectric constant, ε_{r, polymer}, of about 2.3, polyamide may have a dielectric constant, ε_{r, polymer}, of between about 3 and about 4, a butadiene-based copolymer may have a dielectric constant, ε_{r, polymer}, of about 2.5, polyethylene may have a dielectric constant, ε_{r, polymer}, of about 2.3, and polypropylene may have a dielectric constant, ε_{r, polymer}, of about 2.3. In general, it may be preferred that polymer 88 have as high of a dielectric constant as is practical, wherein the dielectric constant may have to be optimized along with mechanical properties of sheath 80 such as flexibility, resiliency, durability, biostability (e.g., biodegradability of sheath 80), resistance from electrical leakage, corrosion resistance, and resistance from damage (e.g., from fracture or tearing).

Filler 90 comprises a material that is mixed in polymer 88 in order to change the effective overall dielectric constant of sheath 80. Filler 90 has a dielectric constant, ε_{r, filler}, that is different from a dielectric constant of polymer 88, ε_{r, polymer}. The weight percentage of filler 90 in sheath 80 may be selected such that an amount of energy induced by an external disruptive energy field in conductor 78 that exceeds a predetermined threshold energy is reflected away from distal end 76. In general, the term “weight percentage of filler 90 in sheath 80” means the percentage of the total weight of sheath 80 that is made up of filler 90. For example, if a particular length of sheath 80 weighs a total of about 1 g and the weight percentage of filler 90 in sheath 80 is about 20 weight %, then the weight of filler 90 would be about 0.2 g. In one example, the dielectric constant of filler 90, ε_{r, filler}, is higher than the dielectric constant of polymer 88, ε_{r, polymer}, such that the overall effective dielectric constant of sheath 80, ε_{r, sheath}, is higher than it would be if sheath 80 comprised polymer 88 alone. For example, the overall effective dielectric constant of sheath 80, resulting from the combination of polymer 88 and filler 90, may be in a range of about 1 to about 10, such as between about 2 and about 5, for example between about 3 and about 4.

In one example, filler 90 comprises small particles or powder that are dispersed within polymer 88. In one example, the particles of filler 90 may be dispersed within polymer 88 or its precursor before polymer 88 is fully cured. For example, if polymer 88 comprises a silicone or another polymer that is formed from two or more constituent parts, then the particles of filler 90 may be mixed within a first constituent of the silicone and a second constituent may be added to the first constituent to initiate reaction of the constituents to form the silicone. In another example, polymer 88 may comprise a polymer that is cured at room temperature or at an elevated temperature, and the particles of filler 90 may be mixed into the precursor constituent or constituents of polymer 88, the mixture may be formed into the desired form factor, such as by extruding the mixture into a generally cylindrical tubing shape over conductor 78, or by molding or casting the mixture into the desired shape of sheath 80. In some examples, the mixture of polymer 88 and filler 90 may be formed into the desired form factor, and then the mixture may be cured, for example by bringing to the mixture to the temperature at which polymer 88 cures.

In some examples, the material of filler 90 has a higher dielectric constant, ε_{r, filler}, than the dielectric constant ε_{r, polymer} of polymer 88. In some examples, the material of filler 90 increases the overall effective dielectric constant of sheath 80, ε_{r, sheath} above the value of ε_{r, polymer}. The dielectric constant of filler 90, ε_{r, filler}, may be as high as is practical in order to maximize the potential effective dielectric constant of sheath 80, ε_{r, sheath}. In some examples, filler 80 comprises a material having a dielectric constant, ε_{r, filler}, of at least about 25, such as a material having a dielectric constant, ε_{r, filler}, of at least about 50, for example a material having a dielectric constant, ε_{r, filler}, of at least about 150, such as a material having a dielectric constant, ε_{r, filler}, of at least about 500. In some examples, filler 80 comprises a material having a dielectric constant, ε_{r, filler}, of between about 4 and about 6000, for example a material having a dielectric constant, ε_{r, filler}, of between about 50 and about 500, such as a material having a dielectric constant, ε_{r, filler}, of between about 150 and about 350. Examples of materials that may be useful as filler 90 include, but are not limited to, silicon dioxide (SiO₂, εᵣ of about 3.9), silicon (εᵣ of about 11.7), aluminum oxide (Al₂O₃, εᵣ of about 9.3 or about 11.5 depending on the crystal structure), titanium dioxide (TiO₂, εᵣ of between about 50 to about 170 depending on the crystal structure), calcium titanate (CaTiO₃, εᵣ of about 180), strontium titanate (SrTiO₃, εᵣ of between about 90 and about 350 depending on the crystal structure), barium-strontium titanate (BST) (Ba₁₋ₓSrₓTiO₃ (for example, wherein x is between about 0 and about 0.3), εᵣ of about 500), barium titanate (BaTiO₃, εᵣ of about 1250 or greater depending on the particle size), and lead zirconate titanate (PZT) (Pb[ZrₓTi₁₋ₓ]O₃ (wherein x is between 0 and 1, for example Pb_{1.1}(Zr_{0.3}Ti_{0.7})O₃), εᵣ of between about 500 and about 6000, depending on the formula and the crystal structure). In some examples, filler 90 comprises a material that is biocompatible, so that if polymer 88 breaks down and some of the particles of filler 90 are released into patient 12, filler 90 will not have an adverse effect.

The overall composition of sheath 80 may be selected in order to optimize the overall configuration of lead 70 such that an amount of energy induced by a disruptive energy field in conductor 78 that is absorbed by tissue of patient 12 proximate distal end 76 is below a predetermined threshold energy. In particular, the weight percentage of filler 90 may be selected such that the characteristic impedance of lead 70 for propagated energy induced at the frequency of a disruptive energy field such as, for example, the fields generated by medical imaging technology such as MRI, is such that much of the propagated energy is reflected from distal end 76 back toward proximal end 74 and the medical device coupled to proximal end 74.

In some examples, the weight percentage of filler 90 in sheath 80 is selected such that an amount of energy induced by an external disruptive energy field in conductor 78 that is absorbed by the target tissue proximate distal end 76 is below a predetermined threshold energy. In other words, the weight percentage of filler 90 in sheath 80 may be selected such that an amount of energy induced by an external disruptive energy field in conductor 78 that exceeds the predetermined threshold energy is reflected away from distal end 76 rather than being absorbed by the target tissue. The value of the predetermined threshold energy, e.g., the value of the propagated induced energy that may be acceptably absorbed by the target tissue, such that any energy above the predetermined threshold that is propagated toward distal end 76 will be reflected back down lead 70 and propagated toward proximal end 74, may depend on several factors, such as the surface area of electrodes 84, 86 that are exposed to the target tissue, and the expected rise in tissue temperature for the target tissue per amount of energy absorbed by the target tissue. For example, as the exposed surface area of electrodes 84, 86 becomes smaller, the predetermined threshold that may be acceptable also becomes smaller, because there is less surface area proximate distal end 76 to dissipate the amount of energy being absorbed from electrodes 84, 86 into the target tissue. This smaller surface area means that a larger amount of energy per surface area will need to be dissipated by electrodes 84, 86, resulting in a larger increase in temperature at electrodes 84, 86. Similarly, as the tendency of the particular target tissue to increase in temperature increases, the predetermined threshold that may be acceptable becomes smaller. This is so because it would take a smaller amount of energy absorbed by the target tissue to increase the temperature of the target tissue by the same amount.

In one example, the predetermined threshold energy value is such that the temperature of the target tissue proximate distal end 76, and particularly the temperature of the target tissue proximate to each electrode 84, 86, increases by less than a threshold temperature rise above the normal temperature of the target tissue so that excessive heating of the target tissue may be avoided. In one example, wherein electrodes 84, 86 are implanted at or near a human patient's heart, the normal temperature at the human heart is about 37° C. (about 99° F.). The threshold temperature rise that may be acceptable may depend on where electrodes 84, 86 will be located. For example, if electrodes 84, 86 are located within a blood vessel or at a location where blood will be perfusing over electrodes 84, 86, then a higher threshold temperature rise may be acceptable because the perfusing blood may provide for efficient and relatively fast cooling of the heated target tissue. In examples wherein electrodes 84, 86 are implanted within a blood vessel or within the heart, an acceptable threshold temperature rise may be up to about 5° C. (e.g., so that the temperature of the target tissue less than about 42° C.). However, in examples wherein electrodes 84, 86 are not implanted where blood will be perfusing, than a smaller temperature rise may be acceptable, such as, for example, less than about 2° C. (e.g., so that the temperature of the target tissue is less than about 39° C.) over a nominal body temperature of the target tissue.

The weight percentage of filler 90 within sheath 80 affects the overall effective dielectric constant of sheath 80, ε_{r, sheath}. In turn, the overall effective dielectric constant of sheath 80, ε_{r, sheath}, affects the characteristic impedance of lead 70. Depending on the frequency of the disruptive energy field, the characteristic impedance of lead 70 may cause induced energy that is propagated along conductor 78 toward distal end 76 to be reflected at distal end 76 such that the induced energy, or at least a substantial portion of it, is reflected from distal end 76 and propagated toward proximal end 74, where it may be dissipated as heat at the medical device coupled to proximal end 74. In general, because filler 90 comprises a material that has a higher dielectric constant, ε_{r, filler}, than that of polymer 88, ε_{r, polymer}, such as TiO₂, filler 90 tends to increase the overall effective dielectric constant of sheath 80, ε_{r, sheath}. In some examples, the increase in overall dielectric constant ε_{r, sheath} causes lead 70 as a whole to become a poorer conductor of propagated energy at high frequencies, such as the frequencies associated with MRI fields.

In some examples, the weight percentage of filler 90 within sheath 80 may be selected so that an overall characteristic impedance of lead 70 is such that an amount of energy induced by an external disruptive energy field in conductor 78 that is absorbed by the target tissue proximate distal end 76 is below the predetermined threshold energy. In some examples, a desired characteristic impedance may be achieved when the weight percentage of filler 90 in sheath 80 is less than about 35 weight % filler 90, such as when the weight percentage of filler 90 in sheath 80 is less than about 20 weight % filler 90, for example when the weight percentage of filler 90 in sheath 80 is less than about 15 weight % filler 90. The weight percentage of filler 90 that may be acceptable in sheath 80 may depend on several factors, including the effect of the weight percentage of filler 90 on mechanical properties of sheath 80 (e.g., on the resulting strength, flexibility, resiliency, durability, biostability, and resistance from damage of sheath 80), the desired characteristic impedance of lead 70 (which in turn depends on the dielectric constant of filler 90, ε_{r, filler}, and the dielectric constant of polymer 88 of sheath 80, ε_{r, polymer}), and the cost of the particular materials of filler 90 used.

Other characteristics of the material of filler 90 may also be selected in order to tune the overall dielectric constant of sheath 80, ε_{r, sheath}, and thus the overall characteristic impedance of sheath 80. In some examples, the size of the particles of filler 90 may be selected in order to achieve a desired overall dielectric constant of sheath 80, ε_{r, sheath}. As noted above, the particle size of the particles of filler 90 may affect the dielectric constant of filler 90, ε_{r, filler}, and also may affect the maximum weight percentage of filler 90 that may be acceptable without adversely affecting the desired mechanical properties of sheath 80. In one example, the particles of filler 90 may have an average particle size, e.g., the length of the largest dimension of the particles, of between about 0.05 micrometers and about 100 micrometers, such as between about 0.5 micrometers and about 75 micrometers, for example between about 20 micrometers and about 50 micrometers. In one example, particles of filler 90 have a size of about 0.06 micrometers. In another example, particles of filler 90 have a size of about 0.5 micrometers.

In some examples, a lead for a medical device comprises a medical lead body having a proximal end for electrically connecting to a medical device and a distal end implantable proximate a target tissue, the lead body comprising a conductor extending from the proximal end to the distal end and a sheath disposed over the conductor, the sheath comprising a polymer and a filler mixed in the polymer, wherein the filler has a dielectric constant that is different from a dielectric constant of the polymer, wherein the weight percentage of the filler in the sheath is selected such that an amount of energy induced by an external disruptive energy field in the conductor that exceeds a predetermined threshold energy is reflected away from the distal end of the lead. In one example, the filler comprises up to about 35 weight % of the sheath. In one example, the filler comprises particles having an average particle size of between about 0.05 micrometers and about 100 micrometers.

In some examples, a system comprises a medical device and a medical lead having a proximal end electrically coupled to the medical device and a distal end implantable proximate a target tissue, the lead comprising a conductor and a sheath disposed over the conductor, the sheath comprising a polymer and a filler mixed in the polymer, wherein the filler has a dielectric constant that is different from a dielectric constant of the polymer, wherein the weight percentage of the filler in the sheath is selected such that an amount of energy induced by an external disruptive energy field in the conductor that exceeds a predetermined threshold energy is reflected away from the distal end of the lead. In one example, the filler comprises up to about 35 weight % of the sheath. In one example, the filler comprises particles having an average particle size of between about 0.05 micrometers and about 100 micrometers.

Although only a single conductor 78 is shown in FIGS. 3 and 4, in other examples, the lead may comprise a plurality of conductors to provide for the transmission of electrical energy to multiple electrodes located on the lead and/or to provide for simultaneous sensing from multiple electrode locations on the lead. FIG. 5 shows an example lead 92 comprising a central body 94, which may comprise an insulating material, such as silicone, silicone rubber, or polyurethane. Central body 94 comprises a plurality of lumens 96, 98, 100, 102, wherein a conductor may be passed through each lumen 96, 98, 100, 102. In one example, lumens 96, 98, 100 each house a cable conductor 104 while lumen 102 houses a coiled conductor 106. Each cable conductor 104 comprises a plurality of fine conductive wires 108, where each cable conductor 104 substantially fills a corresponding lumen 96, 98, and 100. Coiled conductor 106 comprises at least one wire wound into a generally helical coil. In one example, coiled conductor 104 is a multifilar coiled conductor comprising a plurality of coiled conductors 110A, 110B, wherein each coiled conductor 110A, 110B is generally helical and is coaxial, for example wherein alternating courses of coiled conductor 106 comprise a different one of the plurality of coiled conductors 110A, 110B that are interposed between one another. Coiled conductors 110A, 110B provide for a coaxial lumen 112 passing therethrough, which may allow for the insertion of another body, such as a stylet, through lead 92. As shown in FIG. 5, lead 92 provides for a plurality of multiaxial conductors 104, 106 that extend along lead 92, e.g. with each conductor following a different axial path. Each conductor 104, 106 may be made from the same materials as described above for conductor 78 of lead 70.

Lead body 94 may be surrounded by a common sheath 114, while each individual conductor may be surrounded by separate sheaths. For example, as shown in FIG. 5, cable conductors 104 are each surrounded by an individual sheath 116, while coiled conductor 106 is surrounded by individual sheath 118. Lead body 94 and each sheath 114, 116, 118 may comprise an insulative material, such as the materials described above for polymer 88, and one or more of lead body 94 and sheaths 114, 116, 118 may further comprise a filler mixed within the material of the sheath 114, 116, 118 in accordance with the present disclosure. In the example shown in FIG. 5, outer sheath 114 comprises a plurality of particles of filler 120 mixed therein and sheath 118 surrounding coiled conductor 106 comprises a plurality of particles of filler 122 mixed therein, similar to particles of filler 90 mixed within polymer 88 of sheath 80 described above with respect to FIGS. 3 and 4. Filler 120 and filler 122 may comprise the same materials as described above with respect to filler 90. Particles of filler 120 and particles of filler 122 may comprise the same material or mixture of materials, or particles of filler 120 and particles of filler 122 may comprise different materials or mixtures of materials.

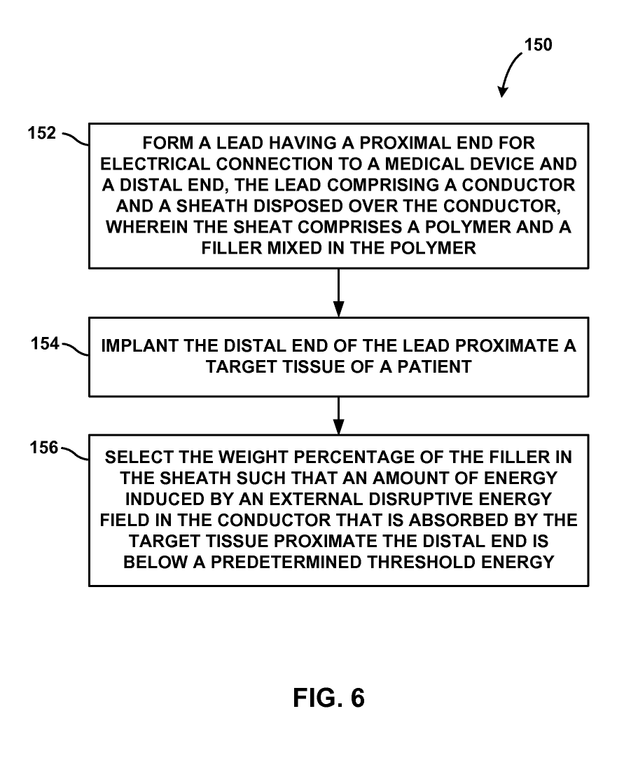

FIG. 6 is a flow diagram of an example method 150 comprising forming a lead 70 having a proximal end 74 for electrical connection to a medical device, such as IMD 16, and a distal end 76 (152), the lead 70 comprising a conductor 78 and a sheath 80 disposed over the conductor 78. The sheath 80 comprises a polymer 88 and a filler 90 mixed in the polymer 90, wherein filler 90 comprises at least one of titanium dioxide, silicon dioxide, silicon, aluminum oxide, calcium titanate, strontium titanate, barium-strontium titanate (BST), barium titanate, and lead zirconate titanate. Method 150 may also comprise implanting distal end 76 of lead 70 proximate a target tissue of a patient (154). As part of forming lead 70 (152), the method 150 may comprise selecting the weight percentage of filler 90 in sheath 80 such that an amount of energy induced by an external disruptive energy field in conductor 78 that is absorbed by the target tissue proximate distal end 78 is below a predetermined threshold energy (156). In some examples, selecting the weight percentage of filler 90 (156) may comprise selecting the weight percentage of filler 90 such that an amount of energy induced by an external disruptive energy field in conductor 78 that exceeds a predetermined threshold energy is reflected away from distal end 76.

In some examples, the method comprises forming a medical lead having a proximal end for electrical connection to a medical device and a distal end implantable proximate a target tissue, the lead comprising a conductor and a sheath disposed over the conductor, the sheath comprising a polymer and a filler mixed in the polymer, wherein the filler has a dielectric constant that is different from a dielectric constant of the polymer, and selecting the weight percentage of the filler in the sheath such that energy induced by an external disruptive energy field in the conductor that is absorbed by the target tissue proximate the distal end of the lead is below a predetermined threshold energy. In one example, the filler comprises up to about 35 weight % of the sheath. In one example, the filler comprises particles having an average particle size of between about 0.05 micrometers and about 100 micrometers.

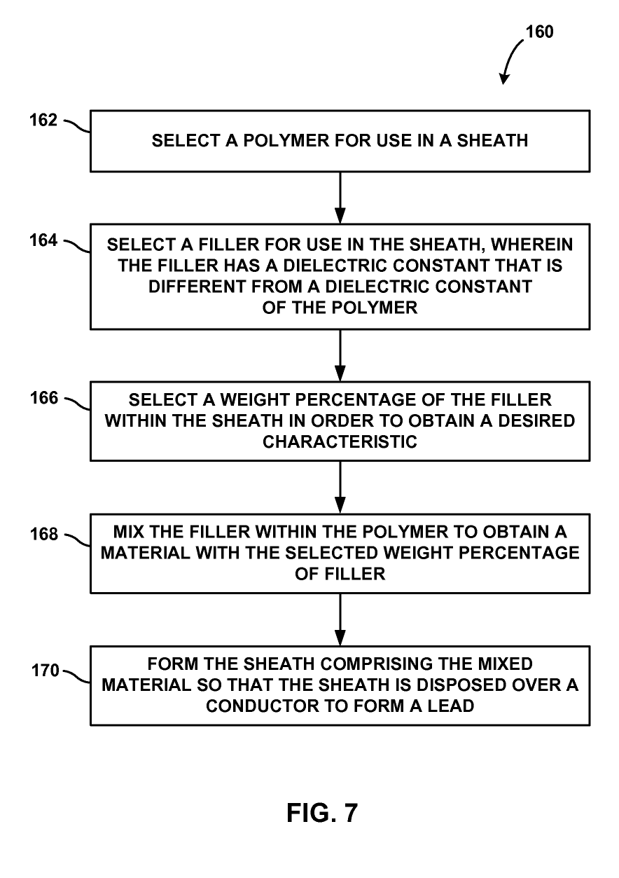

FIG. 7 is a flow diagram of an example method 160 comprising selecting a polymer 88 for use in a sheath 80 (162), selecting a filler 90 for use in the sheath 80, wherein filler 90 has a dielectric constant that is different from a dielectric constant of polymer 88 (164), and selecting a desired weight percentage of filler 90 within sheath 80 in order to obtain a desired characteristic (166). In one example, the characteristic that is desired may be at least one of the overall effective dielectric constant of sheath 88, the characteristic electrical impedance of a lead 70 that sheath 80 is a part of, or an amount of energy induced by an external disruptive energy field in a conductor 78 enclosed by sheath 80 that is absorbed by a target tissue of the patient proximate a distal end 76 of lead 70, e.g., such that the energy absorbed by the target tissue is below a predetermined threshold energy. The method 160 may also comprise mixing filler 90 within polymer 88 to obtain a material with the selected weight percentage of filler 90 (168) and forming sheath 80 comprising the mixed material so that sheath 80 is disposed over conductor 78 to form a lead 70 (170). In one example, forming sheath 80 may be separate from disposing sheath 80 over conductor 78, e.g., extruding the mixed material of sheath 80 to form a tubing followed by inserting conductor 78 through the tubing.

### EXAMPLES

### Example 1

A sample of material that could be used for sheath 80 of a lead 70 was prepared. The sample comprised the ethylene-vinyl acetate copolymer resin sold under the trade name ELVAX 240 by E.I. du Pont de Nemours Co., Wilmington, Del., USA with about 20 weight percent of a titanium (IV) dioxide (TiO₂, εᵣ of approximately 50) mixed therein (e.g., about 20 percent of the weight of sheath 80 comprises titanium dioxide, with the remaining about 80 percent of the weight of sheath 80 comprising ELVAX 240). The mixture was formed into a generally flat sheet that was about 45 millimeters long, about 45 millimeters wide, and about 0.6 millimeters thick. The sheet was used as the dielectric material between conductive plates of a parallel-plate capacitor. An inductance-capacitance-resistance (LCR) meter (4263A LCR Meter sold by Hewlett-Packard/Agilent Technologies, Santa Clara, Calif., USA) was used to measure the resulting capacitance of the parallel-plate capacitor. The capacitance of the parallel-plate capacitor using a dielectric material comprising Elvax 240 and about 20 weight % TiO₂ was measured three times. The resulting capacitance measurements, respectively, were about 111 picofarads, about 107 picofarads, and about 112 picofarads (average capacitance reading of about 110 picofarads).

The capacitance of a parallel-plate capacitor is directly proportional to the dielectric constant, εᵣ, of the dielectric material between the parallel plates, as shown by Equation 1.

$$C = {ɛ}_{r} {ɛ}_{o} \frac{A}{d} \qquad [ 1 ]$$

where C is the capacitance, εᵣ is the dielectric constant of the material between the parallel plates, εₒ is the electric constant (ε₀≈8.854×10⁻¹² F m⁻¹), A is the area of overlap between the parallel plates (in this example about 2025 mm² (45 mm×45 mm), or about 0.00203 m²), and d is distance between the parallel plates (in this example, about 0.6 millimeters). Solving Equation 1 for the dielectric constant, εᵣ, results in Equation 2.

$${ɛ}_{r} = \frac{C \cdot d}{A \cdot {ɛ}_{0}} \qquad [ 2 ]$$

Using Equation 2 and the average capacitance reading of 110 picofarads, it was calculated that the resulting dielectric constant, εᵣ, for the material of Example 1 (80 weight % Elvax 240 and 20 weight % TiO₂ mixed therein) is about 3.67.

### Comparative Example 2

A control sample comprising only Elvax 240 with no filler mixed therein was made into a second sheet. The second sheet had approximately the same dimensions as the first sheet of Example 1 (e.g., about 45 millimeters long by about 45 millimeters wide by about 0.6 millimeters thick). The second sheet was used as the dielectric in a parallel-plate capacitor, and the capacitance was measured three times using the same Hewlett-Packard/Agilent 4263A LCR meter as in Example 1. The resulting capacitance measurements, respectively, were about 85 picofarads, about 85 picofarads, and about 84 picofarads (average capacitance reading of about 84.7 picofarads). The average capacitance of Comparative Example 2 was measured at about 85 picofarads. Using Equation 2, the dielectric constant, εᵣ, of the Elvax 240 was calculated to be about 2.83. Thus, the inclusion of 20 weight % TiO₂ in Example 1 increased the effective dielectric constant of the Elvax 240 by about 30% (from about 2.83 to about 3.67).

The change in capacitance between Example 1 and Comparative Example 2 may also be used to determine an expected difference in a characteristic impedance of a lead made from a sheath comprising the material of Example 1 and a sheath comprising the material of Comparative Example 2. Equation 3 shows the relationship between characteristic impedance, Z₀, and capacitance, C.

$${Z}_{0} = \sqrt{\frac{L}{C}} \qquad [ 3 ]$$

Where C is the capacitance per unit length of the lead, L is the inductance per unit length of the lead, and Z₀ is the characteristic impedance of the line. Assuming that the inductance of the lead of Example 1 compared to the inductance of the lead of Comparative Example 2, the approximately 30% increase in capacitance from Comparative Example 2 to Example 1, discussed above, results in approximately a 15% decrease in the characteristic impedance of the lead of Example 1 compared to the lead of Comparative Example 2.

Various examples have been described. These and other examples are within the scope of the following claims.

1. A method comprising:
forming a medical lead having a proximal end for electrical connection to a medical device and a distal end implantable proximate a target tissue, the lead comprising a conductor and a sheath disposed over the conductor, the sheath comprising a polymer and a filler mixed in the polymer, wherein the filler has a dielectric constant that is different from a dielectric constant of the polymer; and
selecting the weight percentage of the filler in the sheath such that energy induced by an external disruptive energy field in the conductor that is absorbed by the target tissue proximate the distal end of the lead is below a predetermined threshold energy.

2. The method of claim 1, wherein the filler comprises a material having a dielectric constant of at least about 25.

3. The method of claim 1, wherein the filler comprises at least one of titanium dioxide, silicon dioxide, silicon, aluminum oxide, calcium titanate, strontium titanate, barium-strontium titanate (BST), barium titanate, and lead zirconate titanate (PZT).

4. The method of claim 1, wherein the filler comprises a material that is biocompatible.

5. The method of claim 1, wherein the dielectric constant of the filler is larger than the dielectric constant of the polymer.

6. The method of claim 1, wherein the predetermined threshold energy is selected so that a temperature of the target tissue proximate the distal end does not exceed a threshold temperature rise.

7. The method of claim 6, wherein the threshold temperature rise comprises about5° C. over a selected body temperature of the target tissue.

8. The method of claim 1, wherein the polymer of the sheath comprises at least one of a polyurethane, a polyamide, a polyester, a silicone, a silicone rubber, a polyisoprene, a butadiene-based polymer, a butadiene-based copolymer, a polyethylene, a polypropylene, and an ethylene-vinyl acetate copolymer.

9. The method of claim 1, wherein forming the sheath of the lead comprises mixing the filler in a first constituent part of the polymer followed by combining the first constituent part with a second constituent part to form the polymer.

10. The method of claim 1, wherein forming the sheath of the lead comprises mixing the filler with an uncured precursor followed by curing the precursor to form the polymer.

11. A lead for a medical device, the lead comprising:
a conductor having a proximal end for electrically connecting to a medical device and a distal end implantable proximate a target tissue; and
a sheath disposed over the conductor, the sheath comprising a polymer and a filler mixed in the polymer,
wherein the filler has a dielectric constant that is different from a dielectric constant of the polymer;
wherein the weight percentage of the filler in the sheath is selected such that an amount of energy induced by an external disruptive energy field in the conductor that exceeds a predetermined threshold energy is reflected away from the distal end of the lead.

12. The lead of claim 11, wherein the filler comprises a material having a dielectric constant of at least about 25.

13. The lead of claim 11, wherein the filler comprises at least one of titanium dioxide, silicon dioxide, silicon, aluminum oxide, calcium titanate, strontium titanate, barium-strontium titanate (BST), barium titanate, and lead zirconate titanate (PZT).

14. The lead of claim 11, wherein the filler comprises a material that is biocompatible.

15. The lead of claim 11, wherein the dielectric constant of the filler is larger than the dielectric constant of the polymer.

16. The lead of claim 11, wherein the predetermined threshold energy is selected so that a temperature of the target tissue proximate the distal end does not exceed a threshold temperature rise.

17. The lead of claim 16, wherein the threshold temperature rise comprises about 5° C. over a selected body temperature of the target tissue.

18. The lead of claim 11, wherein the polymer of the sheath comprises at least one of a polyurethane, a polyamide, a polyester, a silicone, a silicone rubber, a polyisoprene, a butadiene-based polymer, a butadiene-based copolymer, a polyethylene, a polypropylene, and an ethylene-vinyl acetate copolymer.

19. A system comprising:
a medical device; and
a medical lead having a proximal end electrically coupled to the medical device and a distal end implantable proximate a target tissue, the lead comprising:
a conductor; and
a sheath disposed over the conductor, the sheath comprising a polymer and a filler mixed in the polymer, wherein the filler has a dielectric constant that is different from a dielectric constant of the polymer;
wherein the weight percentage of the filler in the sheath is selected such that an amount of energy induced by an external disruptive energy field in the conductor that exceeds a predetermined threshold energy is reflected away from the distal end of the lead.

20. The system of claim 19, wherein the filler comprises a material having a dielectric constant of at least about 25.

21. The system of claim 19, wherein the filler comprises at least one of titanium dioxide, silicon dioxide, silicon, aluminum oxide, calcium titanate, strontium titanate, barium-strontium titanate (BST), barium titanate, and lead zirconate titanate (PZT).

22. The system of claim 19, wherein the filler comprises a material that is biocompatible.

23. The system of claim 19, wherein the dielectric constant of the filler is larger than the dielectric constant of the polymer.

24. The system of claim 19, wherein the predetermined threshold energy is selected so that a temperature of the target tissue proximate the distal end does not exceed a threshold temperature rise.

25. The system of claim 24, wherein the threshold temperature rise comprises about 5° C. over a selected body temperature of the target tissue.

26. The system of claim 19, wherein the polymer of the sheath comprises at least one of a polyurethane, a polyamide, a polyester, a silicone, a silicone rubber, a polyisoprene, a butadiene-based polymer, a butadiene-based copolymer, a polyethylene, a polypropylene, and an ethylene-vinyl acetate copolymer.
